(19) United States
(12) Patent Application Publication
(10) Pub. No.: US 2022/0409817 A1
(43) Pub. Date: Dec. 29, 2022

(54) SYRINGES, ASSEMBLIES, AND METHODS OF MANUFACTURE

(71) Applicant: AMGEN INC., Thousand Oaks, CA (US)

(72) Inventors: Rasmus Ohlenschlaeger, Hellerup (DK); Jerome Olivas, Thousand Oaks, CA (US)

(21) Appl. No.: 17/846,310

(22) Filed: Jun. 22, 2022

Related U.S. Application Data
(60) Provisional application No. 63/214,218, filed on Jun. 23, 2021.

(51) Int. Cl. A61M 5/31; A61M 5/32

(52) U.S. Cl. CPC A61M 5/3129; A61M 5/3202

(57) ABSTRACT

Syringes, assemblies for use in a filling process, and related methods of manufacture are disclosed. A syringe may include a reservoir filled or configured to be filled with a preselected volume of a drug, including, for example, a preselected volume of 3 mL or approximately 3 mL of the drug. The syringe may further include a wall comprising a cylindrical portion. The cylindrical portion of the wall may include: (a) an inner diameter equal to or approximately equal to a standard inner diameter of a 5 mL syringe or a standard outer diameter of any other syringe volume not equal to the preselected volume or 3 mL; and/or (b) an outer diameter equal to or approximately equal to a standard outer diameter of a 5 mL syringe or a standard outer diameter of any other syringe volume not equal to the preselected volume or 3 mL.

### FIELD OF DISCLOSURE

The present disclosure generally relates to drug delivery devices, and, more particularly, devices for injecting a drug into a patient and the manufacture of such devices.

### BACKGROUND

Syringes are commonly used in the medical field to administer a drug to a patient via an injection, and have other uses and purposes. Syringes are often produced in large quantities and involve multiple suppliers, each being specialized in manufacturing or assembling a different part(s) or component(s) of the syringe. For example, one supplier may manufacture a barrel and/or stopper of the syringe, whereas another supplier may manufacture the drug and/or, in the case of a prefilled syringe, fill the barrel with the drug under, for example, sterile conditions.

The industry has adopted standard dimensions for syringes in order to facilitate coordination among the various suppliers, among other reasons. This standardization has made the production of syringes more efficient and as a result has lowered costs for patients. For example, certain standard syringe dimensions are specified by the International Organization for Standardization (ISO) 11040-4. A consequence of this standardization is that various syringe components are readily available only in certain dimensions. This, in turn, can impose constraints on, for example, manufacturers and the design of a device intended to incorporate the syringe.

The present disclosure sets forth syringes, assemblies, and related methods of manufacture embodying advantageous alternatives to existing syringes, assemblies, and methods of manufacture, and that may address one or more of the challenges or needs mentioned herein, as well as provide other benefits and advantages.

### SUMMARY

One aspect of the present disclosure provides a syringe including at least a reservoir and a wall. The wall may include at least a cylindrical portion. The reservoir may be filled or configured to be filled with a preselected volume of a drug, including, for example, a preselected volume of 3 mL or approximately 3 mL of the drug. The cylindrical portion may include: (a) an inner diameter equal to or approximately equal to a standard inner diameter of a 5 mL syringe or a standard outer diameter of any other syringe volume not equal to the preselected volume or 3 mL; and/or (b) an outer diameter equal to or approximately equal to a standard outer diameter of a 5 mL syringe or a standard outer diameter of any other syringe volume not equal to the preselected volume or 3 mL. In some embodiments, ISO 11040-4 may indicate the standard inner diameter of a 5 mL syringe and/or the standard outer diameter of a 5 mL syringe.

The syringe may have a first length measured between a proximally facing inner surface of the wall and a proximally facing outer surface of the wall. The first length of the syringe may be less than a standard length of a 3 mL syringe or 72.2 mm; and/or the first length of the syringe may be equal to or approximately equal to a standard length of a 1 mL syringe or 54 mm. In some embodiments, ISO 11040-4 may indicate the standard length of a 3 mL syringe and/or the standard length of a 1 mL syringe.

Another aspect of the present disclosure provides a method of manufacturing a syringe having a preselected volume, including, for example, a preselected volume of 3 mL. The method may include providing or obtaining a specification of standard syringe dimensions, wherein the specification indicates for each of a plurality of syringe volumes at least: (a) one of a plurality of standard diameters, and (b) one of a plurality of standard lengths. The specification of standard syringe dimensions may be included in, for example, ISO 11040-4. The method may further include selecting a standard diameter from among the plurality of standard diameters included in the specification, wherein the syringe volume indicated by the specification for the selected standard diameter does not equal the preselected volume. In some embodiments, the syringe volume indicated by the specification for the selected standard diameter may be 5 mL.

A further aspect of the present disclosure provides an assembly for use in at least a filling process. The assembly may include a tub and a plurality of syringes each disposed at least partially in the tub. One or more of the syringes may include a reservoir filled or configured to be filled with a preselected volume of 3 mL of a drug or approximately 3 mL of the drug. Furthermore, one or more of the syringe may have a length that is less than a standard length for a 3 mL syringe. In some embodiments, the tub may have a height equal to or approximately equal to 3 inches or 76.2 mm.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicative of the presence or absence of particular elements in any of the exemplary embodiments, except as may be explicitly delineated in the corresponding written description. Also, none of the drawings is necessarily to scale.

FIG. 1 is a chart of standard dimensions for a syringe as specified by ISO 11040-4.

FIG. 2 is a perspective view of an exemplary syringe in accordance with various embodiments.

FIG. 3 is a side view of the syringe in FIG. 2.

FIG. 4 is a cross-sectional view taken along longitudinal axis A of the syringe in FIG. 3.

FIG. 5 is a top view of a flange of the syringe in FIG. 4, with the stopper omitted.

FIG. 6 is a perspective view of the stopper of the syringe in FIGS. 2-5.

FIG. 7 is a side view of the stopper in FIG. 6.

FIG. 8 is a cross-section view taken along longitudinal axis A of the stopper in FIG. 7.

FIG. 9 is an exploded perspective view of an exemplary assembly for use in at least a filling process.

FIG. 10 is a non-exploded perspective view of the assembly in FIG. 9.

### DETAILED DESCRIPTION

The present disclosure generally relates to the design and manufacture of syringes. The syringes disclosed herein may deviate in at least some respect(s) from the standard dimensions used for manufacturing syringes, including, for example, those specified by ISO 11040-4, in order to, for example, achieve a desired syringe design, accommodate existing manufacturing processes, and/or satisfy externally imposed design constraints such as volume and/or length requirements needed for incorporating the syringe into an automated drug delivery device such as an autoinjector and/or on-body injector and/or ergonomic considerations. The way(s) in which the presently disclosed syringes deviate from standard dimensioning are chosen strategically so as to minimize or eliminate any potential customization or changes to standard manufacturing equipment or practices to be employed in producing the presently disclosed syringes. This streamlines the process of manufacturing the presently disclosed syringes and potentially broadens the group of suppliers from which it is feasible to source component(s) for manufacturing the presently disclosed syringes. For example, in order to achieve a syringe having a desired volume and/or a desired length, the present disclosure describes choosing a standard diameter which a specification of standard syringe dimensions, such as ISO 11040-4, does not associate with the desired volume and/or the desired length. This may allow one to utilize, for example, a standard tubing cane, which may be readily available from various suppliers in standard diameters, to manufacture the syringe with the desired volume and/or the desired length. These and other advantages and benefits will be apparent to one of ordinary skill in the art reviewing the present disclosure.

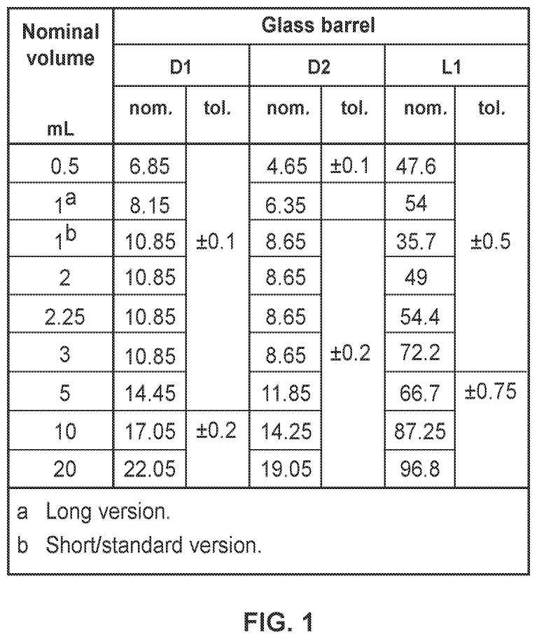

FIG. 1 illustrates a chart of standard dimensions for a syringe as specified by ISO 11040-4. The entire contents of ISO 11040-4 are incorporated by reference herein. The chart in FIG. 1 specifies various standard dimensions (measured in millimeters (mm)) for each of a plurality nominal volumes (measured in milliliters (mL)) of a syringe. In the chart, D1 corresponds to an outer diameter of a barrel (e.g., a glass barrel) of a syringe, D2 corresponds to an inner diameter of the barrel of the syringe, and L1 corresponds to the length of the barrel of the syringe. The chart indicates, for example, that a syringe having a nominal volume of 5 mL (sometimes referred to as a “5 mL syringe”) should be manufactured with a barrel (e.g., a glass barrel) having: a standard outer diameter D1 of 14.45 mm±0.1 mm, a standard inner diameter D2 of 11.85 mm±0.2 mm, and a length L1 of 66.7 mm±0.75 mm. As another example, the chart indicates that a syringe having a nominal volume of 3 mL (sometimes referred to as a “3 mL syringe”) should be or must be manufactured with a barrel (e.g., a glass barrel) having: a standard outer diameter D1 of 10.85 mm±0.1 mm, a standard inner diameter D2 of 8.65 mm±0.2 mm, and a length L1 of 72.2±0.5 mm. As a further example, the chart indicates that the “long version” of a syringe having a nominal volume of 1 mL (commonly referred to as a “1 mL syringe”) should be manufactured with a barrel (e.g., a glass barrel) having: a standard outer diameter D1 of 8.15 mm±0.1 mm, a standard inner diameter D2 of 6.35 mm±0.1 mm, and a length L1 of 54 mm±0.5 mm.

The inventors of the present disclosure have found that the standard barrel lengths specified by, for example, ISO 11040-4, may be undesirable and/or unacceptable for certain applications. For example, it may be medically necessary for a certain drug to be injected in a 3 mL dose; however, a drug delivery device (e.g., an autoinjector or on-body injector) to be used in conjunction with a syringe including the 3 mL dose may not be configured to house or otherwise accommodate a syringe having a barrel length L1 equal to or larger than 72.2±0.5 mm, which is the barrel length L1 specified by ISO 11040-4 for a 3 mL syringe. As a more specific example, a barrel length equal to or approximately equal to the barrel length L1 specified by ISO 11040-4 for a 1 mL syringe may be appropriate (e.g., from an engineering perspective) for a particular application requiring the 3 mL syringe. Rather than re-design the drug delivery device to accommodate the barrel length L1 specified by ISO 11040-4 for a 3 mL syringe, or, alternatively, design a customized syringe which completely ignores the standard dimensions specified by ISO 11040-4, the inventors of the present application have selected various standard syringe dimension(s) to arrive at a novel combination of standard syringe dimension(s) and/or non-standard syringe dimension(s) which advantageously meets the dimensional requirement(s) of a particular application (e.g., use of the syringe in an autoinjector or an on-body injector or manufacturing process) while nevertheless minimizing or eliminating any potential customization or changes to standard or existing manufacturing equipment or practices to be employed in producing the syringe. As an example, a 3 mL syringe according to an embodiment of the present disclosure may be manufactured with the standard outer diameter D1 and/or the standard inner diameter D2 specified by ISO 11040-4 (or another specification of standard syringe dimensions) for a 5 mL syringe and with a barrel length L1 that is less than the standard barrel length L1 specified by ISO 11040-4 (or another specification of standard syringe dimensions) for a 3 mL syringe, including, for example, a barrel length L1 equal to or approximately equal to the standard barrel length L1 specified by ISO 11040-4 (or another specification of standard syringe dimensions) for a 1 mL syringe.

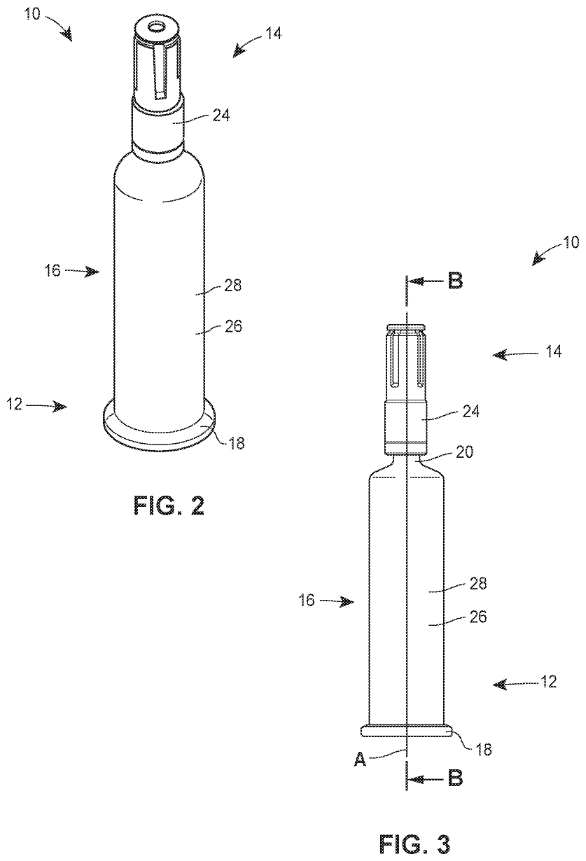

FIGS. 2-5 illustrate an embodiment of a syringe 10 constructed in accordance with principles of the present disclosure. Generally the syringe 10 may be configured for delivering (for example, injecting) a drug (which may also be referred to herein as a medicament or drug product) to, for example, a patient. The drug may be, but is not limited to, various biologics such as peptides, peptibodies, and/or antibodies. The drug may be in a fluid or liquid form, gelatin form, and/or powdered form, although the present disclosure is not limited to a particular form or state of the drug.

The syringe 10 may include a longitudinal axis A and a proximal end 12 and a distal end 14 arranged at respective positions along the longitudinal axis A. The proximal end 12 of the syringe 10 may include, for example, a barrel 16 (e.g., a body portion) and/or a flange 18. The distal end 14 of the syringe 10 may include, for example, a neck 20, a needle 22, and/or a removable sterile barrier 24. The syringe 10 may include a wall 26 defining any one or any combination of: the barrel 16, the flange 18, and the neck 20. As an example, the wall 26 may include a cylindrical portion 28 defining at least a portion of, or the entirety of, the barrel 16. The syringe 10 may additionally include a stopper 32 (also referred to in some contexts as a “plunger” or “plunger stopper”) disposed partially or entirely within barrel 16.

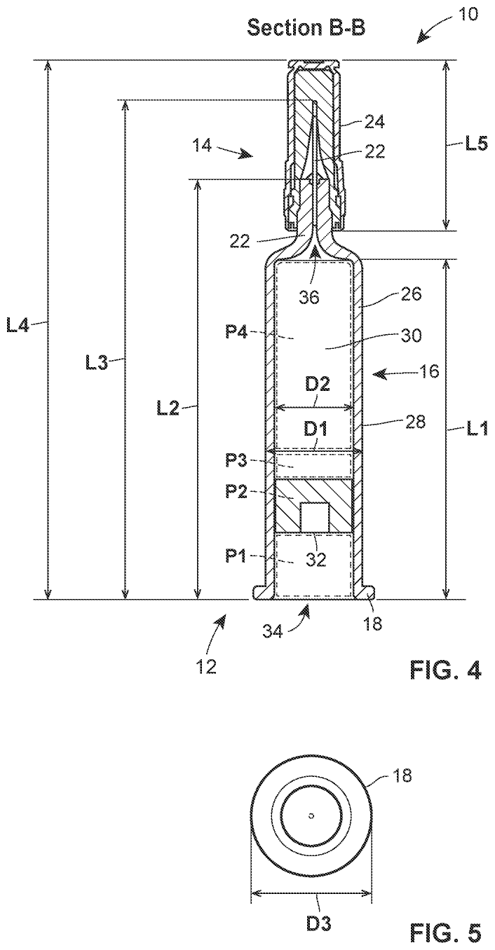

As seen in FIG. 4, an interior space of the barrel 16 may include at least a first portion P1, a second portion P2, a third portion P3, and/or a fourth portion P4. Each of the portions P1-P4 may correspond to a three-dimensional space having a preselected length measured along the longitudinal axis A and/or which is necessary to perform a desired function. As an example: the first portion P1 may correspond to a portion of the interior space of the barrel 16 that is proximal to the stopper 32; the second portion P2 may correspond to a portion of the interior space of the barrel 16 occupied by the stopper 32 prior to use of the syringe 10; the third portion P3 may correspond to a portion of the interior space of the barrel 16 that is distal to the stopper 32 and provides a gap (e.g., an air gap) between the stopper 32 and the drug when the syringe 10 is arranged in an upright orientation prior to use; and/or the fourth portion P4 may correspond to a portion of the interior space of the barrel 16 that is filled partially or entirely with the drug when the syringe 10 is arranged in an upright orientation prior to use.

The syringe 10 may additionally include a reservoir 30 filled or configured to be filled, partially or entirely, with a preselected volume (e.g., a predetermined volume, an intended volume, and/or a maximum intended volume) of a drug. As an example, the reservoir 30 may correspond to at least a portion of the interior space of the barrel 16 and/or at least a portion of the interior space of the neck 20. As a more specific example, the reservoir 30 may correspond the fourth portion P4 of the interior space of the barrel 16. As an even more specific example, the preselected volume of the reservoir 30 and/or the preselected volume of the drug in the reservoir 30 may correspond to a volume of at least the fourth portion P4 of the interior space of the barrel 16. As another example, the preselected volume of the reservoir 30 and/or the preselected volume of the drug in the reservoir 30 may correspond to the sum of a volume of the fourth portion P4 of the interior space of the barrel 16 and a volume of an interior space of the neck 20.

The reservoir 30 of the syringe 10 may be defined partially or entirely by a distally facing surface of the stopper 32 and an inner surface of barrel 16 (e.g., an inner surface of the cylindrical portion 28 of the wall 26) and/or neck 20.

The reservoir 30 may be filled partially or entirely with a drug. In some embodiments, the reservoir 30 may be prefilled with a drug, for example, by a manufacturer; whereas, in other embodiments, the reservoir 30 may be provided empty such that a downstream manufacturer or user is required to fill the reservoir 30 with a drug. The preselected volume of the reservoir 30 and/or the preselected volume of the drug in the reservoir 30 may be equal to or approximately (e.g., ±10% or ±5%) equal to any of the “nominal volumes” included in the chart in FIG. 1. As an example, the reservoir 30 and/or the drug therein may have a preselected volume equal to or approximately (e.g., ±10% or ±5%) equal to 3 mL.

The wall 26 of the syringe 10 may be made partially or entirely of a rigid or semi-rigid material including, for example, glass, plastic (e.g., polypropylene), and/or any other suitable material or combination of materials.

The stopper 32 may be movably disposed within the barrel 20 such that it can move at least in a distal direction along the longitudinal axis A from an initial position adjacent to a proximal end of the barrel 16 to at least an end-of-dose or end-of-delivery position adjacent to a distal end of the barrel 16. Distal movement of the stopper 32 may expel the drug from the syringe 10 via the needle 22. Proximal movement of the stopper 32 along the longitudinal axis A may also be possible in at least some embodiments. The stopper 32 may be constructed of an elastomeric material such as rubber or any other suitable material. In some embodiments, the stopper 32 may be coated partially or entirely with a fluoropolymer film (e.g., a FluroTec® barrier film). The stopper 32 may slidably and/or sealingly contact the inner surface of the barrel 16 such that, for example, the drug in the reservoir 30 is prevented or inhibited from leaking past the stopper 32 when the stopper 32 moves in the distal direction. As an example, the stopper 32 may form a fluid-tight and/or air-tight seal with the inner surface of the cylindrical 28 of the wall 26.

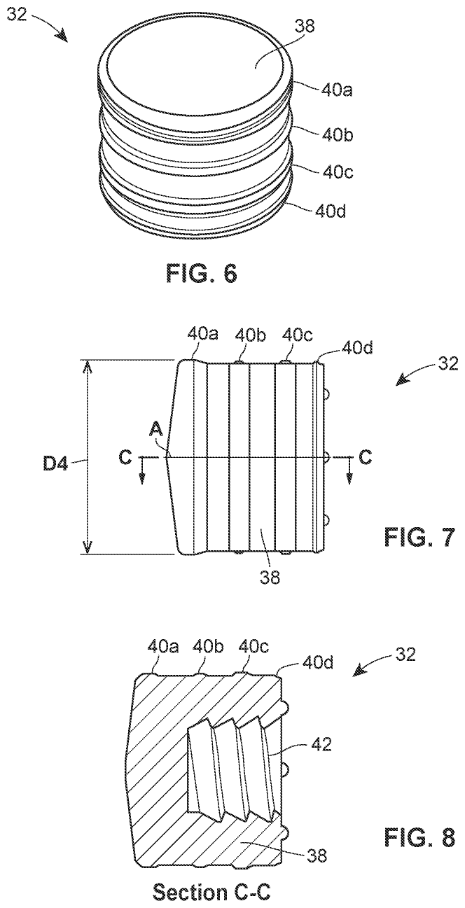

The stopper 32 may include a generally cylindrical body portion 38 and one or more ribs 40a-d extending radially outwardly from the body portion 38, as seen in FIG. 6-8. At least an outwardly facing surface of one or more of the ribs 40a-d may contact the inner surface of the barrel 16. Each of the ribs 40a-d may partially or entirely surround the body portion 38 of the stopper 32. The ribs 40a-d may be spaced apart from each another along a direction that is parallel to the longitudinal axis A. The cylindrical body portion 38 may include a cavity or opening to facilitate coupling the stopper 32 to, for example, a plunger rod. As seen in FIG. 8, this cavity may be surrounded to a threaded inner surface 42 of the stopper 32. As an example, the threaded inner surface 42 may be configured to threadably couple to a threaded outer surface of a plunger rod.

The proximal end of the barrel 16 may include a proximal axial opening 34 permitting a plunger rod or other drive element to extend into the barrel 16 and couple with and/or move the stopper 32 with respect to the wall 26. The neck 20 may include a distal axial opening 36 configured to provide fluid communication with the drug in the reservoir 30. As an example, the needle 22 may be coupled with the distal end of the neck 20 and/or in fluid communication with the reservoir 30 via the distal axial opening 36. As a more specific example, the needle 22 may fixed (e.g., adhered and/or staked) to the portion of the wall 26 defining the neck 20 such that the needle 22 cannot move with respect to the wall 26. A distal end of the needle 22 may include a sharpened tip or other pointed geometry allowing the distal end of the needle 22 to pierce and/or penetrate through a patient's skin, subcutaneous tissue, and/or other tissue. The needle 22 may be hollow and/or include an axial passage that is parallel to and/or coaxial with the longitudinal axis A of the syringe 10. One or more openings may be formed in the distal end of the needle 22 to allow drug to flow out of the needle 22 into the patient during use of the syringe 10. The needle 22 may be made of metal and/or any other suitably rigid material. In alternative embodiments, the needle 22 may be omitted (such that the syringe 10 is considered, for example, a needless syringe) or at least not directly coupled with the wall 26 of the syringe 10. In certain such alternative embodiments, the neck 20 may be coupled with and/or form a nozzle or other fluid path member including, for example, a Luer Lock fitting. In embodiments where the needle 22 is omitted, the neck 20 may also be omitted and a distal axial opening in the barrel 16 may be covered with a septum or other structure capable of providing selective fluid communication with the reservoir 30.

As seen in FIG. 4, the removable sterile barrier 24 may be coupled with the neck 22 and/or cover the distal end of the needle 22. The removable sterile barrier 24 may be configured to form an air-tight and/or fluid-tight seal with the neck 22 so as to provide a sterile or otherwise clean environment for storing the needle 22 prior to use of the syringe 10. Immediately or substantially immediately prior to use of the syringe 10, the removable sterile barrier 24 may be removed from the neck 22 (e.g., by a user) to expose the distal end of the needle 22. As an example, the removable sterile barrier 24 may be a rigid needle shield (RNS) or a non-rigid needle shield (nRNS).

With reference to FIGS. 4-8, the dimensioning of the syringe 10 will now be described. The cylindrical portion 28 of the wall 26 of the syringe 10 may have an outer diameter D1 and an inner diameter D2. As an example, the outer diameter D1 and/or the inner diameter D2 of the cylindrical portion 28 of the wall 28 may be constant or substantially constant along the longitudinal axis A. A portion or the entirety of the barrel 16 may also possess the outer diameter D1 and/or the inner diameter D2. In terms of what they measure, the outer diameter D1 and the inner diameter D2 may correspond to, respectively, the outer diameter D1 and the inner diameter D2 specified by ISO 11040-4 (as described above in conjunction with FIG. 1). The flange 18 may have an outer diameter D3.

The syringe 10 may further include various lengths, some or all of which may be measured in a direction parallel or substantially parallel to the longitudinal axis A. As seen in FIG. 4, the syringe 10 may have a length L1 (referred to as a “barrel length” in some contexts) measured between a proximally facing outer surface of the wall 26 and a proximally facing portion of the inner surface of the wall 26. In some embodiments, the length L1 may correspond to the length of the cylindrical portion 28 of the wall 26 and/or the length of the barrel 16. In terms of what is measured by the length L1, the length L1 may correspond to the length L1 specified by ISO 11040-4 (as described above in connection with FIG. 1). Additionally, the syringe 10 may have a length L2 measured between the proximally facing outer surface of the wall 26 and a distally facing outer surface of the wall 26. The latter may, in some embodiments, correspond to a distally facing outer surface of the neck 20 of the syringe 10. The syringe 10 may further include a length L3 measured between the proximally facing outer surface of the wall 26 and a distally facing surface of the needle 22. Additionally, the syringe 10 may include a length L4 measured between the proximally facing outer surface of the wall 26 and a distally facing outer surface of the removable sterile barrier 24. The syringe 10 may also include a length L5 measured between a proximally facing outer surface of the removable sterile barrier 24 and the distally facing outer surface of the removable sterile barrier 24.

As seen FIG. 7, the stopper 32 may include an outer diameter D4. As an example, the outer diameter D4 may correspond to an outer diameter of one or more of the ribs 40a-d of the stopper 32. Furthermore, the stopper 32 may have a length L6. As seen in FIG. 8, the length L6 may be measured between a distally facing outer surface of the stopper 32 and a proximally facing outer surface of the stopper 32.

The values of one or more of the diameters D1-D4, one or more of the lengths L1-L6, and/or other dimensions of the syringe 10 may be selected to provide a novel combination of dimensions that achieves multiple objectives and/or balances competing needs. For example, the dimensions may be selected in order to provide a syringe having a design that: (a) is configured to store a preselected (e.g., predetermined, targeted, desired, etc.) volume of a drug; (b) has ergonomic benefits; (c) can be manufactured using standard (or mostly standard) manufacturing practices and/or equipment; (d) can be made from a standard tubing cane (e.g., a standard glass tubing cane); (e) can be transported in a standard syringe tub; (f) accommodates the structural and/or functional requirements of a drug delivery device such as, for example, an autoinjector or on-body injector; and/or (g) minimizes deviations from standard syringe dimensions including, for example, those specified by ISO 11040-4.

As an example, in a scenario where the syringe 10 has a volume that is preselected (e.g., due to the required dosage of a drug to be administered by the syringe 10), a method of manufacturing the syringe 10 according to an embodiment of the present disclosure may include one or more of the following steps. Initially, one may provide or obtain a specification of standard syringe dimensions which indicates for each of a plurality of syringe volumes any one or any combination of: a standard outer diameter D1, a standard inner diameter D2, and a standard length L1. The specification of standard syringe dimensions may be included in, for example, ISO 11040-4. Next, one may select a standard outer diameter D1, a standard inner diameter D2, a standard length L1, and/or another standard dimension for which the specification does not associate with the preselected volume but, for example, does associate with a syringe volume not equal to the preselected volume. For example, if the preselected volume is 3 mL, one may select a standard outer diameter D1 and/or a standard inner diameter D2 which the specification associates with a 5 mL syringe. In doing so, one may have the flexibility to, for example, select a length L1 for the syringe 10 that is less than or more than the standard length L1 that the specification associates with a 3 mL syringe, without, for example, compromising the ability to manufacture the syringe 10 from a standard tubing cane (e.g., a standard tubing cane conventionally used for manufacturing 5 mL syringes). For example, one may select a length L1 which is equal to or approximately (e.g., ±10% or ±5%) equal to the standard length L1 which the specification associates with a 1 mL syringe. As another example, one may calculate the selected length L1 according to a mathematical formula wherein the selected length L1 is equal to or approximately (e.g., ±10% or ±5%) equal to:

$$\frac{Preselected Volume}{( π ) * {( \frac{Selected Standard Diameter}{2} )}^{2}}$$

As another example, the syringe 10 may be designed to include any one or any combination of the following: (a) the reservoir 30 containing or configured to contain 3 mL of a drug or approximately (e.g., ±10% or ±5%) 3 mL of a drug such that, in at least some contexts, the syringe 10 is considered to be a “3 mL syringe”; (b) the cylindrical portion 28 of the wall 26 and/or the barrel 16 having an outer diameter D1 equal to or approximately (e.g., ±10%, ±5%, or ±0.1 mm) equal to 14.45 mm; (c) the cylindrical portion 28 of the wall 26 and/or the barrel 16 having an inner diameter D2 equal to or approximately (e.g., ±10%, ±5%, or ±0.1 mm) equal to 11.85 mm; (d) the flange 18 having an outer diameter D3 equal to or approximately (e.g., ±10%, ±5%, or ±0.35 mm) equal to 18 mm; (e) the cylindrical portion 28 of the wall 26 and/or the barrel 16 having a length L1 equal to or approximately (e.g., ±10%, ±5%, or ±0.5 mm) equal to 54 mm or 35.7 mm; (f) the wall 26 having a length L2 equal to or approximately (e.g., ±10%, ±5%, or ±0.5 mm) equal to 62.3 mm; (g) a length L3 equal to or approximately (e.g., ±10%, ±5%, or ±1 mm) equal to 75 mm; (h) a length L4 equal to or approximately (e.g., ±10%, ±5%, or ±1 mm) equal to 80 mm; (i) a length L5 equal to or approximately (e.g., ±10%, ±5%, or ±0.3 mm) equal to 25.1 mm; (j) the stopper 32 having a length L6 equal to or approximately (e.g., ±10%, ±5%, or ±0.3 mm) equal to 10 mm; and/or (k) the stopper 32 having an outer diameter D4 equal to or approximately (e.g., ±10%, ±5%, or ±0.1 mm) equal to 12.4 mm, with or without any coating applied to the exterior of the stopper 32. Any one or any combination of dimensions (a)-(k) mentioned in the preceding sentence may be preselected.

Manufacturing the syringe 10 with an outer diameter D1 and/or inner diameter D2 that is larger than what is specified by, for example, ISO 11040-4, for a given volume may allow the syringe 10 to have a shorter length L1 than what is specified by, for example, ISO 11040-4, for the given volume. As a result, it may be possible for one to select a length L1 for the syringe 10 that is suitable for use with a standard syringe tub, including, for example, a syringe tub having height H equal to or approximately (e.g., ±10% or ±5%) equal to 3 inches (i.e., 76.2 mm). This may enable or facilitate processing of the syringe 10 at many or most existing fill sites or other existing manufacturing facilities because, for example, standard filling machines and/or other standard manufacturing equipment used at such facilities may be equipped for handling standard syringe tubs, including, for example, a syringe tub having a height H of 3 inches. As an example, a 3 mL version of the syringe 10 if designed, as discussed above, to include a standard outer diameter D1 and/or a standard inner diameter D2 as specified by, for example, ISO 11040-4, for a 5 mL syringe, may have a length L1 equal to or approximately (e.g., ±10%, ±5%, or ±0.5 mm) equal to 54 mm, and, as a result of this length L1, may be transported, stored, and/or processed along with other similarly dimensioned syringes in a syringe tub having a height H of 3 inches.

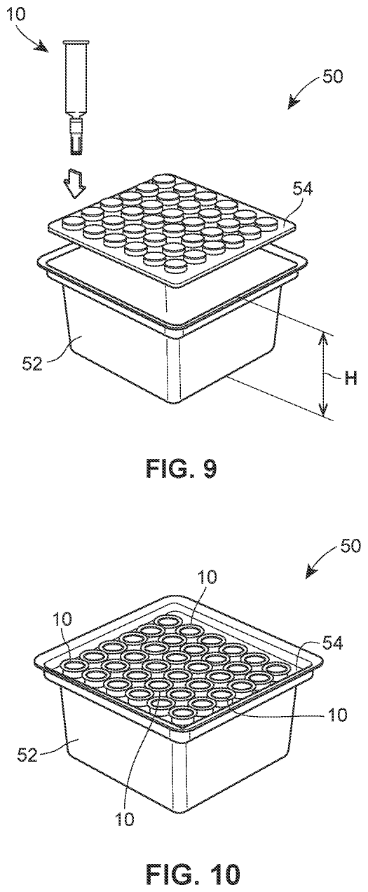

FIGS. 9-10 illustrate an example of an assembly 50 including a plurality of the syringes 10 and a syringe tub 52. The syringe tub 50 may have a plurality of wells receiving respective syringes 10 and/or may orient the syringes 10 such that the longitudinal axes A1 of the syringes 10 are parallel or substantially parallel to each other and/or a vertical direction. As an example, the assembly 50 may include a tray 54 whose outer periphery rests on a ledge located at a top end of the syringe tub 52 and which includes a plurality of openings defining the wells. At least a portion of the distal end 14 of each syringe 10 may be inserted into a respective one of the wells, as seen in FIGS. 9-10. At least a portion of the proximal end 16 of each syringe 10 (including, e.g., the flange 18) may be disposed above the tray 54 and/or above the opening in the top of the syringe tub 52, as seen in FIG. 10. As a result, the proximal axial opening 34 of each syringe 10 may be accessible from above, thereby enabling, for example, a fluid dispenser of a filling machine to fill the barrel 16 of the syringe 10 with a drug from above. The syringe tub 52 illustrated in FIGS. 9-10 is configured to hold thirty six (36) syringes 10; but other configurations of the syringe tub 52 may be configured to hold any suitable number of syringes 10. In some embodiments, the entire assembly 50, or at least a portion thereof, may be enclosed within medical grade (e.g., sterile) packaging.

The syringe tub 52, when viewed from above, may have a generally rectangular shape, square shape, or any other suitable shape. Furthermore, the syringe tub 52 may possess a height H measured between, for example, a bottommost surface and a topmost surface of the syringe tub 52. As an example, the height H may be equal to or approximately (e.g., ±10% or ±5%) equal to 3 inches. As mentioned above, such a height H may be compatible with a wide variety of standard filling machines and/or other standard equipment used for manufacturing syringes.

The presently disclosed syringes and methods of manufacturing syringes are not limited to selecting the specific combinations of dimensions explicitly described herein. The syringes according to the present disclosure may be designed and/or manufactured to include any desired combination of standard syringe dimensions (including, for example, the standard syringe dimensions specified in ISO 11040-4) and/or non-standard syringe dimensions.

The syringes according to the present disclosure may be used in any suitable application and/or incorporated into (e.g., installed within) any suitable device. As an example, the syringe 10 may be incorporated into a drug delivery device such as, for example, an autoinjector or an on-body injector. As a more specific example, the syringe 10 may incorporated into any of the autoinjectors and other drug delivery devices described in U.S. patent application Ser. No. 17/036,690, filed Sep. 29, 2020, U.S. patent application Ser. No. 17/035,851, filed Sep. 29, 2020, U.S. patent application Ser. No. 17/035,927, filed Sep. 29, 2020, U.S. patent application Ser. No. 17/036,129, filed Sep. 29, 2020, U.S. patent application Ser. No. 17/036,217, filed Sep. 29, 2020, U.S. Provisional Application No. 63/159,317, filed Mar. 10, 2021, and U.S. Provisional Application No. 63/159,356, filed Mar. 10, 2021, the entire contents of each are incorporated by reference herein.

All features described herein, including in the specification, claims, abstract, and drawings, and all the steps in any method or process described herein, may be combined in any combination, except combinations where one or more of the features and/or steps are mutually exclusive.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RAN KL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like;Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4ß7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFß mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) molecules such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNFα monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)—N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonypethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRAS^{G12C} small molecule inhibitor, or another product containing a KRAS^{G12C} small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BITE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1(PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A syringe comprising:
a reservoir filled or configured to be filled with a preselected volume of 3 mL of a drug or approximately 3 mL of the drug; and
a wall comprising a cylindrical portion, wherein the cylindrical portion comprises at least one of:
an inner diameter equal to or approximately equal to a standard inner diameter of a 5 mL syringe, and
an outer diameter equal to or approximately equal to a standard outer diameter of a 5 mL syringe.

2. The syringe of claim 1, wherein ISO 11040-4 indicates the standard inner diameter of a 5 mL syringe and/or the standard outer diameter of a 5 mL syringe.

3. The syringe of claim 1, wherein the inner diameter of the cylindrical portion of the wall is equal to or approximately equal to 11.85 mm and/or wherein the outer diameter of the cylindrical portion of the wall is equal to or approximately equal to 14.45 mm.

4. (canceled)

5. The syringe of claim 1, wherein the syringe has a first length measured between a proximally facing inner surface of the wall and a proximally facing outer surface of the wall.

6. The syringe of claim 5, wherein the first length of the syringe is either (a) or (b):
(a) less than a standard length of a 3 mL syringe or 72.2 mm, where ISO 11040-4 indicates the standard length of a 3 mL syringe, or
(b) equal to or approximately equal to a standard length of a 1 mL syringe or 54 mm where ISO 11040-4 indicates the standard length of a 1 mL syringe.

7-9. (canceled)

10. The syringe of claim 1, wherein the syringe has a second length measured between a distally facing outer surface of the wall and a proximally facing outer surface of the wall, the second length being equal to or approximately equal to 62.3 mm.

11. (canceled)

12. The syringe of claim 1, wherein the syringe has a needle and the syringe has a third length measured between a distally facing outer surface of the needle and a proximally facing outer surface of the wall, the third length being equal to or approximately equal to 75 mm.

13-14. (canceled)

15. The syringe of claim 12, further comprising a removable sterile barrier covering at least a portion of the needle, and wherein the syringe has a fourth length measured between a distally facing outer surface of the removable sterile barrier and a proximally facing outer surface of the wall, the fourth length being equal to or approximately equal to 80 mm.

16. The syringe of claim 12, wherein the syringe has a fifth length measured between a distally facing outer surface of the removable sterile barrier and a proximally facing outer surface of the removable sterile barrier, the fifth length being equal to or approximately equal to 25.1 mm.

17-20. (canceled)

21. The syringe of claim 1, further comprising a stopper disposed at least partially within the cylindrical portion of the wall, and wherein a sixth length is measured between a distally facing surface of the stopper and a proximally facing surface of the stopper, the sixth length being equal to or approximately equal to 10 mm.

22. A method of manufacturing a syringe having a preselected volume, the method comprising:
providing or obtaining a specification of standard syringe dimensions, wherein the specification indicates for each of a plurality of syringe volumes at least: (a) one of a plurality of standard diameters, and (b) one of a plurality of standard lengths; and
selecting a standard diameter from among the plurality of standard diameters included in the specification, wherein the syringe volume indicated by the specification for the selected standard diameter does not equal the preselected volume.

23. The method of claim 22, wherein the preselected volume is 3 mL.

24. The method of claim 22, wherein the syringe volume indicated by the specification for the selected standard diameter is (a) 5 mL and/or (b) larger than the preselected volume.

25-28. (canceled)

29. The method of claim 26, comprising selecting a length for the syringe, wherein the selected length (a) does not equal the standard length indicated by the specification for the preselected volume, (b) is less than the standard length indicated by the specification for the preselected volume, (c) is equal to or approximately equal to the standard length indicated by the specification for a 1 mL syringe, and/or (d) is calculated according to a mathematical formula wherein the selected length is equal to or approximately equal to:
$$\frac{Preselected Volume}{( π ) * {( \frac{Selected Standard Diameter}{2} )}^{2}} .$$

30. The method of claim 22, wherein the specification of standard syringe dimensions comprises ISO 11040-4.

31. (canceled)

32. The method of claim 22, wherein the plurality of standard diameters correspond to a plurality of standard outer and/or inner diameters of the syringe.

33. (canceled)

34. An assembly for use in at least a filling process, the assembly comprising:
a tub; and
a plurality of syringes each disposed at least partially in the tub, wherein one or more of the syringes comprises:
reservoir filled or configured to be filled with a preselected volume of 3 mL of a drug or approximately 3 mL of the drug, and
a length less than a standard length for a 3 mL syringe.

35. The assembly of claim 34, wherein the tub has a height equal to or approximately equal to 3 inches or 76.2 mm.

36. The assembly of claim 34, wherein one or more of the syringes has an outer and/or inner diameter equal to or approximately equal to a standard outer diameter of a 5 mL syringe.

37. (canceled)

38. The assembly of claim 34, wherein one or more of the syringes syringe has a length equal to or approximately equal to a standard length of a 1 mL syringe.

39. (canceled)
